(19) United States
(12) Patent Application Publication
(10) Pub. No.: US 2017/0119341 A1
(43) Pub. Date: May 4, 2017

(54) HANDHELD INTRA-PATIENT OBJECT DETECTION DEVICE

(71) Applicants: Jeffrey A. Szczepanski, Traverse City, MI (US); Fredrick P. Schoville, Brighton, MI (US)

(72) Inventors: Jeffrey A. Szczepanski, Traverse City, MI (US); Fredrick P. Schoville, Brighton, MI (US)

(21) Appl. No.: 14/929,508

(22) Filed: Nov. 2, 2015

(51) Int. Cl. A61B 8/08; A61B 8/00

(52) U.S. Cl. CPC A61B 8/0833; A61B 8/4416; A61B 8/46; A61B 8/565

(57) ABSTRACT

An apparatus for metal detection is disclosed. The apparatus includes a housing having a first end portion sized and adapted for holding by a user and a second end portion opposite the first end portion, wherein the second end portion includes a centrally located aperture, a detection coil within the second end portion, and a detection circuit connected to the detection coil, the detection circuit and detection coil being configured to detect the presence of an element proximate to the second end portion.

### TECHNICAL FIELD

This disclosure relates to implants, and more particularly to detecting visually hidden or masked items within a patient during surgery.

### BACKGROUND

The statements in this section merely provide background information related to the present disclosure and may not constitute prior art.

There are certain situations in which it may be necessary or desirable to locate a metallic or nonmetallic object or mechanical fastener or other object within the body. For example, many surgical procedures require the temporary implantation of mechanical fasteners such as screws, pins, metal wires, staples, etc. that subsequently must be removed. Implanted screws can require removal due to complications like pain, infection, ineffectiveness, repair or replacement. There are also situations in which it is desirable to remove a fastener after a bone or other structure has healed. Aside from fasteners and other objects that are deliberately implanted, metallic objects—like bullets, shrapnel, and other shards of metal—sometimes make their way into the body and require removal. Many surgical tools and instruments such as clips, clamps, retractors, forceps used during surgery can be misplaced during surgeries and particularly during emergency surgeries requiring time-critical actions.

The position of the fastener, surgical tools, or other metallic or nonmetallic object may be difficult to immediately identify visually during surgery, as they may be obscured by blood or body tissues. Accordingly, there is a need for a handheld object detection device for use during surgery to identify foreign objects within the patient and to allow a surgeon or user of the device to accurately mark a location associated with the detecting on a patient's surface.

### SUMMARY

An apparatus for metal and/or nonmetallic detection is disclosed. The apparatus includes a housing having a first end portion sized and adapted for holding by a user and a second end portion opposite the first end portion, wherein the second end portion includes a centrally located aperture, a detection coil within the second end portion, and a detection circuit connected to the detection coil, the detection circuit and detection coil being configured to detect the presence of an element proximate to the second end portion.

This summary is provided merely to introduce certain concepts and not to identify key or essential features of the claimed subject matter.

### BRIEF DESCRIPTION OF THE DRAWINGS

One or more embodiments will now be described, by way of example, with reference to the accompanying drawings, in which:

FIG. 1 schematically shows an exemplary detection system, in accordance with the present disclosure;

FIG. 2 is a perspective view of the detection device, in accordance with the present disclosure;

FIG. 3 is an exploded view of the detection device, in accordance with the present disclosure;

FIG. 4 is a front view of the detection device, in accordance with the present disclosure;

FIG. 5 is a cross-sectional view of the detection device taken along line A-A of FIG. 4, in accordance with the present disclosure;

FIG. 6 is a top view of the detection device, in accordance with the present disclosure;

FIG. 7 schematically shows the detection device, in accordance with the present disclosure; and

FIG. 8 shows a process for detecting and removing an object during a medical procedure, in accordance with the present disclosure.

### DETAILED DESCRIPTION

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” includes plural reference, and the meaning of “in” includes “in” and “on.” The phrase “in one embodiment,” as used herein does not necessarily refer to the same embodiment, although it may. Similarly, the phrase “in some embodiments,” as used herein, when used multiple times, does not necessarily refer to the same embodiments, although it may. As used herein, the term “or” is an inclusive “or” operator, and is equivalent to the term “and/or,” unless the context clearly dictates otherwise. The term “based upon” is not exclusive and allows for being based on additional factors not described, unless the context clearly dictates otherwise. The term “exemplary” used throughout this description means “serving as an example, instance, or illustration,” and should not necessarily be construed as preferred or advantageous over other exemplary embodiments.

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

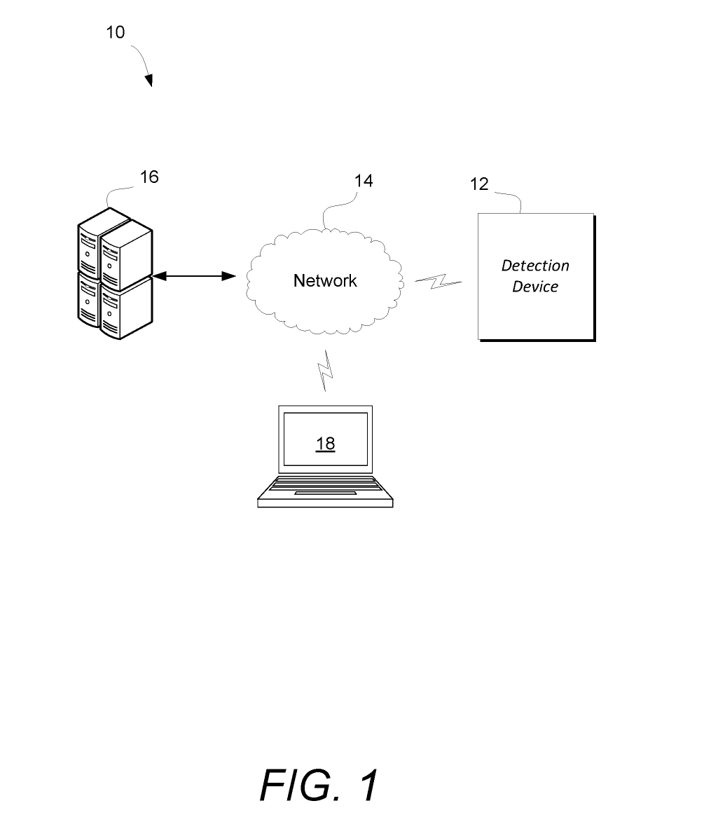

Referring now to the drawings, wherein the depictions are for the purpose of illustrating certain exemplary embodiments only and not for the purpose of limiting the same, FIG. 1 schematically shows an exemplary detection system 10 that may help implement the surgical metal detection apparatus methodologies of the present disclosure. The system 10 includes a computing device 18, a server system 16, a network 14, and a detection device 12. As shown in FIG. 1, the computing device 18 may be directly communicatively connected to the detection device 12 via the network 14 and/or directly communicatively connected to the detection device 12. The server system 16 may be directly communicatively connected to the computing device 18 and the detection device 12 via the network 14. The detection device 12 may be physically connected to the network 14 or the computing device 18 during selected periods of operation without departing from the teachings herein. Components of the system 10 are shown in FIG. 1 as single objects. Such illustration is for ease of description and it should be recognized that the system 10 may include multiple additional mobile and computing devices.

The network 14 may be any suitable series of points or nodes interconnected by communication paths. The network 14 may be interconnected with other networks and contain sub networks network such as, for example, a publicly accessible distributed network like the Internet or other telecommunications networks (e.g., intranets, virtual nets, overlay networks and the like). The network 14 may facilitate the exchange of data between and among the detection device 12, the computing device 18, and the server system 16 although in various embodiments the detection device 12 may be directly connected to the computing device 18.

The computing device 18 and the server system 16 may each be: various embodiments of a computer including high-speed microcomputers, minicomputers, mainframes, and/or data storage devices. The server system 16 preferably executes database functions including storing and maintaining a database and processes requests from the detection device 12 and the computing device 18 to extract data from, or update, a database as described herein below. The server 16 may additionally provide processing functions for the detection device 12 and the computing device 18 as will become apparent to those skilled in the art upon a careful reading of the teachings herein.

In addition, the detection device 12 may include one or more applications that the user may operate. Operation may include downloading, installing, turning on, unlocking, activating, or otherwise using the application. The application may comprise at least one of an algorithm, software, computer code, and/or the like, for example, mobile application software. In the alternative, the application may be a website accessible through the world wide web.

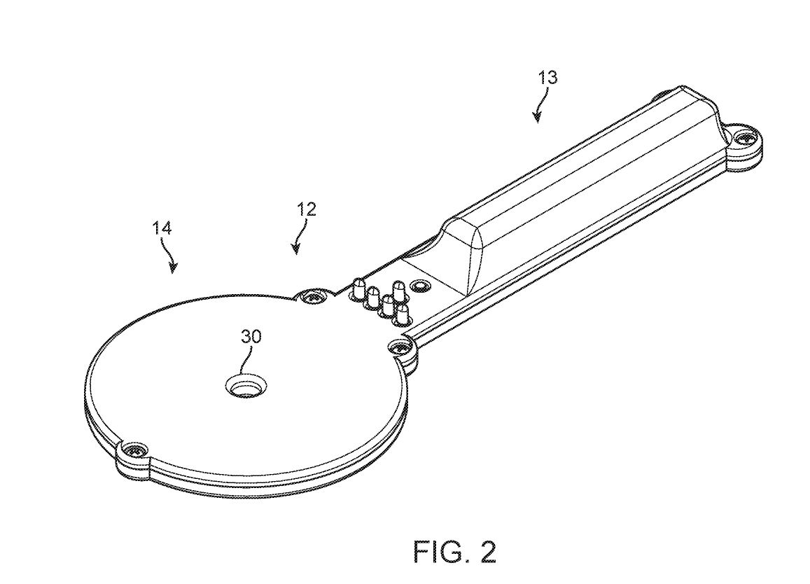

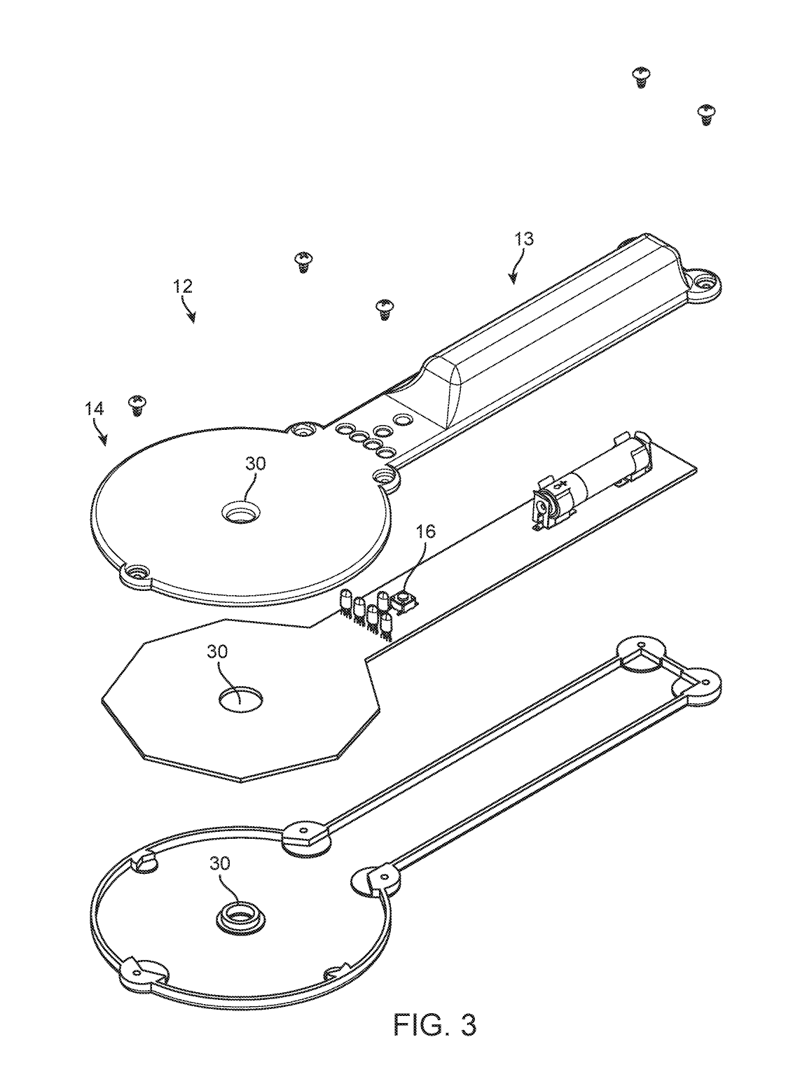

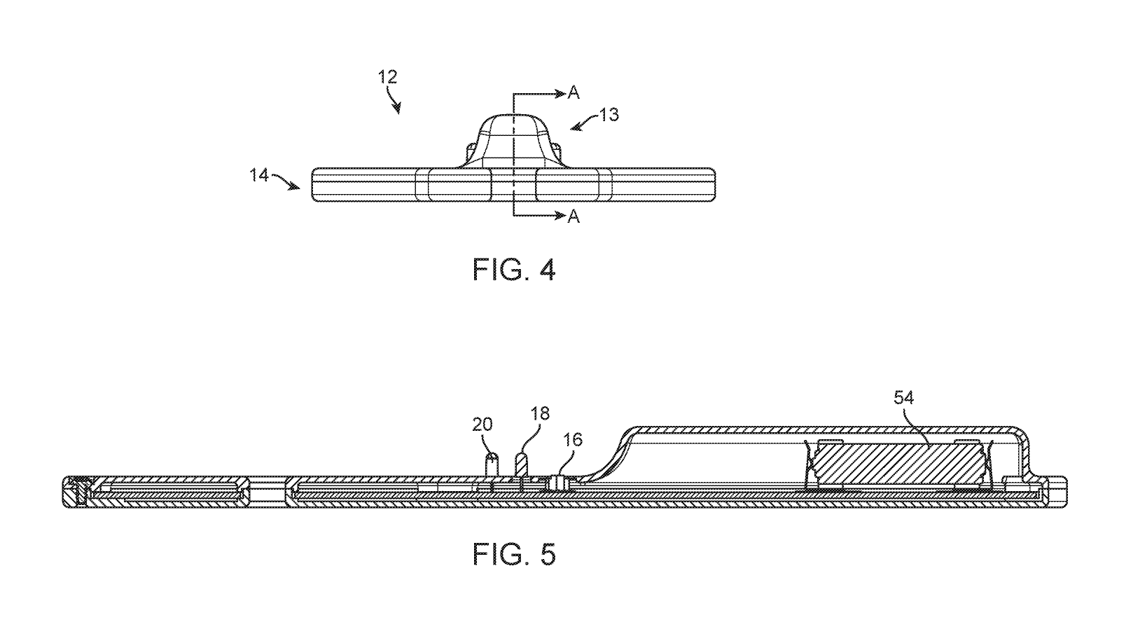

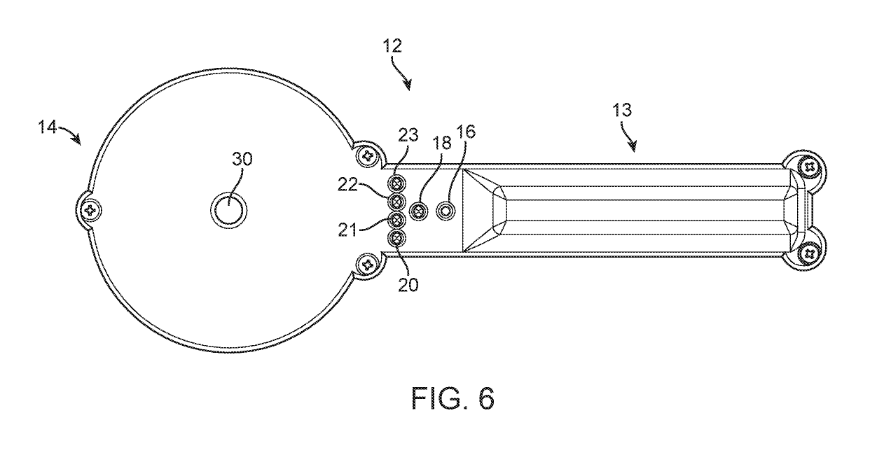

FIGS. 2-6 depict various views of the detection device 12. As FIGS. 2-6 show, the device 12 includes a handle 13 and a planar-shaped detection portion 14. In one embodiment, the handle 13 is ergonomically-shaped. The device 12 includes an ON/OFF switch 16, a first lighting device 18 configured to indicate an ON/OFF operating state, and a plurality of exemplary lighting devices 20, 21, 22, and 23 configured to indicate to a user detection of a foreign object. One skilled in the art will readily acknowledge that one or more types of lighting devices may be incorporated into the device 12 including light emitting diodes and electroluminescent wire.

The device 12 is equipped with an inductance-to-digital converter 40 that measures parallel impedance of an LC resonator 42 located in the detection portion 14. In this way, the device 12 detects objects by receiving energy and not providing energy into a patient that may interfere with embedded medical devices. The planar-shaped portion 14 further includes an aperture 30 from which a medical professional may mark through the device upon detecting a foreign object. For example, an indication may be made to the user such as via the lighting devices 20-23, an audio signal or a display that a foreign object is below the portion 14 of the device 12. The detected indication of a foreign object is weaker at some areas that others, e.g., it is stronger when in close proximity and weaker further away. Upon making a determination that the detected indication is stronger at a particular point, the user may hold the device in that position, insert a pen or marker through the aperture 30 and make a mark on the patient or bandage indicating the area where the foreign object is detected.

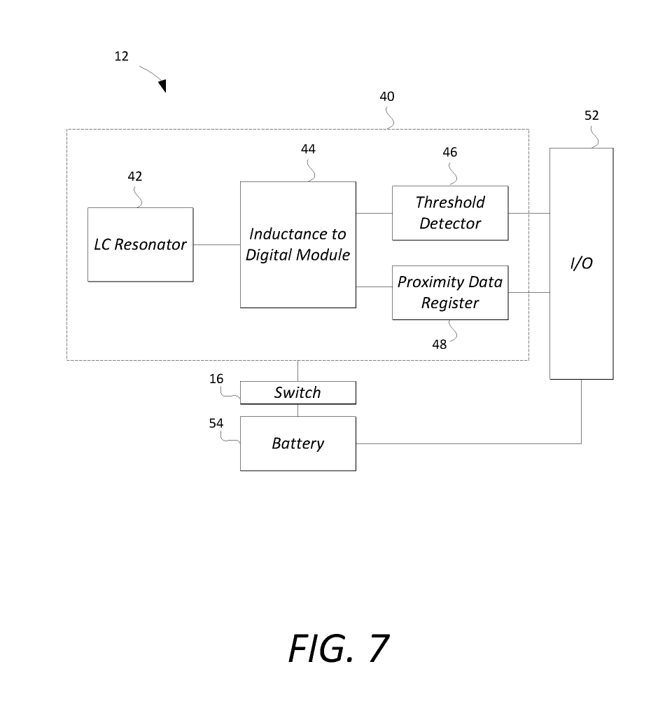

FIG. 7 schematically show an exemplary detection device 12. As FIG. 7 shows, the device 12 includes an inductance-to-digital converter 40 that measures the parallel impedance of an LC resonator 42. In one embodiment, the LC resonator 42 may be housed within the planar-shaped portion 14 as shown in FIGS. 2-5. An inductance-to-digital module 44 measure the parallel impedance of the LC resonator 42 by regulating the oscillation amplitude in a closed-loop configuration to a constant level, while monitoring the energy dissipated by the LC resonator 42. In one embodiment, a threshold detector 46 provides a comparator of the inductance-to-digital module 44 with hysteresis functionality. The inductance-to-digital module 44 may then compare measurements using the threshold detector 46 and a proximity data register 48. In various embodiments, an input/output circuitry 52 for connecting peripheral devices such as the lighting devices 20, 21, 22, and 23 and the switch 16 may be used. A battery or power source 54 is preferably connected to the device 12. In various embodiments, the battery 54 may be connected via the switch 16. The battery may be directly connected to the lighting devices 20, 21, 22, and 23 and/or through the input/output circuitry 52.

In various embodiments, the device 12 may utilize beat frequency oscillation (BFO), Very low frequency (VLF), Pulse induction (PI), dielectric capacitance and/or ultra sound technology and/or single transistor RF pulse circuit, and/or induction balance, and/or, balanced coils, or any combination of these technologies. In operation, the user activates the device 12 with a push button 16, the user physically moves the handheld device 12 over the skin or open surgical field. As the signal strength changes an audible and/or visual indicator, e.g., from the lighting devices 20-23 will alert or otherwise indicate to the user nearing the location of presence of the foreign material.

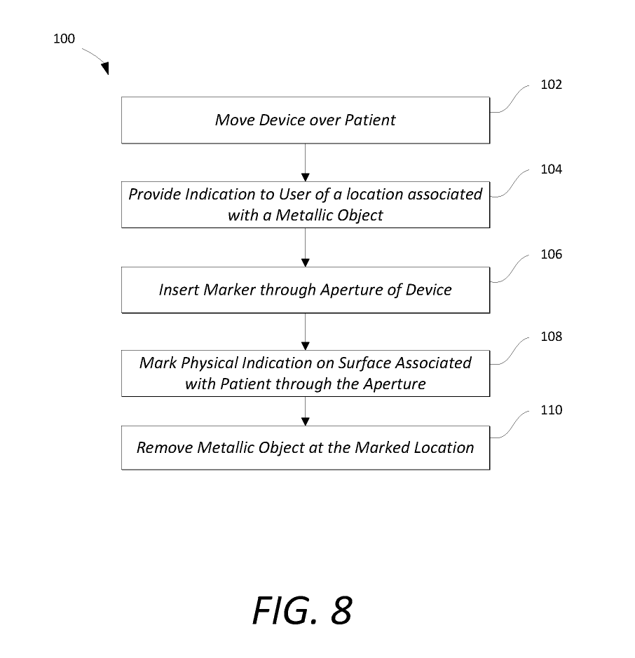

FIG. 8 shows a process 100 for detecting and removing an object during a medical procedure. The process begins at step 102 wherein a surgeon or medical professional moves the device 12 over a patient's body including an area of operation. At step 104, upon detecting an object proximate to or within a patient, the device 12 provides an indication. The indication may be made to the user using one or more of the lighting devices 20-23, using a speaker, or tactile device. In various embodiments, the aperture 30 of the device 12 is the point at which the detection is indicated. Hence, at step 106, a user should insert a marker or other marking device through the aperture 30 of the device 12 at the location indicated by the device 12. At step 108, the user marks a physical indication on a surface associated with the location on the patient or surface material, e.g., bandage, associated with the indicated location. At step 110, the surgeon removes the metal detection device at the marked location.

The disclosure has described certain preferred embodiments and modifications thereto. Further modifications and alterations may occur to others upon reading and understanding the specification. Therefore, it is intended that the disclosure not be limited to the particular embodiment(s) disclosed for carrying out this disclosure, but that the disclosure will include all embodiments falling within the scope of the appended claims.

1. A surgical metal detection apparatus, comprising:
a housing having a first end portion sized and adapted for holding by a user and a second end portion opposite the first end portion, wherein the second end portion includes a centrally located aperture;
a detection coil within the second end portion; and
a detection circuit connected to the detection coil, the detection circuit and detection coil being configured to detect the presence of an element proximate to the second end portion.

2. The surgical metal detection apparatus of claim 1, wherein the detection coil comprises an inductor and a capacitor.

3. The surgical metal detection apparatus of claim 1, wherein the detection circuit comprises an inductance-to-digital module configured to detect metallic and nonmetallic objects.

4. The surgical metal detection apparatus of claim 1, wherein the detection coil is wrapped around a circumference of the second end portion.

5. The surgical metal detection apparatus of claim 1, wherein the aperture is elliptical-shaped.

6. The surgical metal detection apparatus of claim 1, wherein the aperture is substantially perpendicular to a top and bottom portion of the second end portion.

7. The surgical metal detection apparatus of claim 1, wherein the detection circuit is a beat frequency oscillation metal detection circuit.

8. The surgical metal detection apparatus of claim 1, wherein the detection circuit comprises a very low frequency (VLF) circuit.

9. The surgical metal detection apparatus of claim 1, wherein the detection circuit comprises a pulse induction (PI) circuit.

10. The surgical metal detection apparatus of claim 1, wherein the detection circuit comprises a dielectric capacitance component.

11. The surgical metal detection apparatus of claim 1, wherein the detection circuit comprises ultra sound components.

12. The surgical metal detection apparatus of claim 1, wherein the detection circuit comprises a single transistor RF pulse circuit.

13. The surgical metal detection apparatus of claim 1, wherein the detection circuit comprises an induction balance component.

14. A surgical metal detection apparatus, comprising:
a housing having a first end portion sized and adapted for holding by a user and a second end portion opposite the first end portion, wherein the second end portion includes a centrally located aperture;
a detection coil within the second end portion;
a detection circuit connected to the detection coil, the detection circuit and detection coil being configured to detect the presence of an element proximate to the second end portion; and
an indicating component actuatable by detection of the element proximate to the second end portion.

15. The surgical metal detection apparatus of claim 14, wherein the indicating component is a plurality of lighting devices.

16. The surgical metal detection apparatus of claim 14, wherein the indicating component is a speaker.

17. The surgical metal detection apparatus of claim 14, wherein the indicating component is a tactile component.

18. A method of detecting and removing an object during a medical procedure, comprising:
moving an object detection device over a patient;
upon an indication that an object is proximate to an aperture formed of the object detection device at an indicated point,
marking the indicated point, through the aperture of the detection device upon a surface of the patient,
removing the object detection device, and
removing the object at the marked point.

19. The method of claim 18, wherein the indication that the object is proximate to the metal detection device is indicated from one of a lighting device, a speaker and a tactile component.

20. The method of claim 18, wherein the aperture comprises a central indication point of the indication.
